(19) United States
(12) Patent Application Publication
(10) Pub. No.: US 2016/0195551 A1
(43) Pub. Date: Jul. 7, 2016

(54) METHOD FOR EVALUATING SALIVARY ADIPONECTIN LEVEL AND KIT FOR MEASURING SALIVARY ADIPONECTIN LEVEL

(71) Applicants: Kazuhisa MAEDA, Osaka (JP); NIPRO CORPORATION, Osaka-shi, Osaka (JP)

(72) Inventors: Kazuhisa Maeda, Suita-shi, Osaka (JP); Hsiaoyun Lin, Suita-shi, Osaka (JP); Hiroshi Yoshida, Osaka-shi, Osaka (JP); Mitsuhiro Morita, Osaka-shi, Osaka (JP)

(21) Appl. No.: 14/916,183

(22) PCT Filed: Sep. 3, 2014
(86) PCT No.: PCT/JP2014/073269

§ 371 Date: Mar. 3, 2016

(30) Foreign Application Priority Data
Sep. 3, 2013 (JP) 2013-182631
Sep. 3, 2014 (JP) 2014-179682

(51) Int. Cl. G01N 33/74; C07K 16/26

(52) U.S. Cl. CPC G01N 33/74; C07K 16/26; G01N 2333/575; G01N 2800/22

(57) ABSTRACT

A method for evaluating a salivary adiponectin level, comprising evaluating the salivary adiponectin level by the level of multimeric adiponectin of 280 kDa or more. A method for evaluating a salivary adiponectin level, wherein a saliva sample is fractionated into human multimeric adiponectin of 280 kDa or more and human multimeric adiponectin of less than 280 kDa. A kit for measuring a salivary adiponectm level, comprising a reagent of an anti-adiponectin antibody which reacts specifically with multimeric adiponectin of 280 kDa or more in the collected saliva, wherein the salivary adiponectin level is evaluated by the level of the multimeric adiponectin of 280 kDa or more, by the reagent.

### TECHNICAL FIELD

The present invention relates to a method for evaluating a salivary adiponectin level of a human or a biological body that secretes saliva, and a measurement kit for evaluating the adiponectin level. The present invention particularly relates to a method for specifying a secretion amount of adiponectin of a human or a biological body that secretes saliva, and a measurement kit for evaluating the secretion amount of adiponectin. Also, the present invention particularly relates to a method for selecting an anti-adiponectin antibody used in the method for evaluating a salivary adiponectin level.

### BACKGROUND ART

Metabolic syndrome is a complication state of two or more of hypertension, hyperlipemia, and hyperglycemia, in addition to obesity. Severe metabolic syndrome induces arteriosclerosis, and leads to myocardial infarction and cerebral infarction, thus early detection and prevention are regarded as the most important. In our country, specific medical examination and specific health guidance are executed, but are executed once a year, and the like, thus it cannot be said as sufficient as measures for early detection and prevention. For early detection and prevention of metabolic syndrome, it is desirable that each person conducts daily self health management. One of the health management methods includes common monitoring of the level of biomarker that can be a metabolic syndrome index.

As the index of metabolic syndrome, adiponectin, insulin, and the like have been reported, and conventionally, there has been a suggestion relating to a method for measuring adiponectin and/or insulin (Patent Document 1). Adiponectin is a protein secreted from fat cells. Reduction in the adiponectin secretion amount in the blood contributes to insulin resistance insulin responsiveness reduction, and metabolic syndrome outbreak, and causes hyperinsulinemia (increase in the insulin secretion amount in the blood). Accordingly, when the content of adiponectin and the content of insulin in the blood can be monitored, early detection and prevention of metabolic syndrome are also possible.

However, a biological sample used in the monitoring is blood, and thus there is a problem that the blood collection is highly invasive. Also, it is a safety concern for an individual to perform blood collection, and also, blood collection is poor in simplicity. Based on these reasons, the metabolic syndrome index in the blood is hardly monitored, except for some monitoring such as blood sugar self-measurement. It is theoretically possible to monitor with a biological sample other than blood, for example, saliva, since the metabolic syndrome index is also contained in the saliva. It has been revealed that the contents of adiponectin and insulin in the saliva have a correlation with the contents in the blood.

However, monitoring by the saliva has a problem that sensitivity is markedly insufficient. While the reason is not clear, it is expected because measurement interfering impurities are much contained.

A purpose of this conventional suggestion is to develop a technology for sensitively monitoring a biomarker that is the index of so-called metabolic syndrome, with which even an individual without expertise and skills can monitor simply and accurately without requiring skills and which is useful in self health management. Trials and errors are repeated for solving the above object, and in the process, stimulatory saliva is focused, in that the sample is non-invasive and highly safe and can be simply collected, and when a biomarker candidate suitable for monitoring by stimulatory saliva is studied, it has been found out that adiponectin and insulin are useful.

According to this conventional suggestion, the index of metabolic syndrome can be sensitively monitored, thus even an individual without expertise and skills can measure simply and accurately without requiring skills, and due to daily self health management, prevention and early detection of metabolic syndrome are possible.

However, there are cases that saliva is determined to be positive for occult blood (contamination of blood) by blood from periodontal tissue destroyed by gingivitis and periodontal disease, and when compared in the presence or absence of occult blood, a marked difference was found in the concentration of salivary adiponectin between a person who is determined to be positive for occult blood and a person who is determined to be negative for occult blood, and it could be seen that the measurement of salivary adiponectin is affected by the presence or absence of occult blood.

More specifically, regarding the quantification of salivary adiponectin, there has been a problem that the effect of blood contamination cannot be ignored.

### PRIOR ART DOCUMENT

### Patent Document

Patent Document 1: Japanese Patent Laid-open Publication No. 2012-122788

### SUM MARY OF THE INVENTION

### Problems to be Solved by the Invention

Therefore, an object of this invention is to provide a method for evaluating a salivary adiponectin level, which is less affected by the presence or absence of occult blood, and a measurement kit for evaluating the salivary adiponectin level.

### Solutions to the Problems

In order to achieve the object, the following technical means is provided in this invention.

(1) The method for evaluating a salivary adiponectin level of this invention comprises evaluating the salivary adiponectin level by the level of multimeric adiponectin of 280 kDa or more.

The present inventor has found that in the molecular weight distribution of salivary human multimeric adiponectin, high molecular weight (HMW) adiponectin (280 kDa or more) is mainly expressed, unlike the blood in which medium molecular weight (MMW) adiponectin and low molecular weight (LMW) adiponectin of less than 280 kDa are highly expressed.

Moreover, it has been found that adiponectin of a saliva sample determined to be positive for occult blood (contamination of blood) is affected by occult blood, therefore, expression of the medium molecular weight (MMW) adiponectin and low molecular weight (LMW) adiponectin, not the high molecular weight (HMW) adiponectin of 280 kDa or more, increases. Also, the level of salivary multimeric adiponectin of 280 kDa or more has been found to have a correlation with the total salivary adiponectin level.

In the present invention, multimeric adiponectin with a molecular weight of 280 kDa or more (or the amount thereof) is written as “high molecular weight adiponectin” or “HMW”, multimeric adiponectin with a molecular weight of 150 kDa or more and less than 280 kDa (or the amount thereof) is written as “medium molecular weight adiponectin” or “MMW”, and multimeric adiponectin with a molecular weight of 0 kDa or more and less than 150 kDa (or the amount thereof) is written as “low molecular weight adiponectin” or “LMW”. The same applies hereafter.

More specifically, the total adiponectin level detected using a saliva sample consists of the sum of the HMW value, the MMW value, and the LMW value. Here, comparing a saliva sample of the occult blood positive group with contamination of blood with a saliva sample of the occult blood negative group without contamination of blood, the MMW value and the LMW value increase in the sample of the occult blood positive group, but decrease in the sample of the occult blood negative group. Therefore, for example, when adiponectin is evaluated by the total adiponectin level as conventional, the sum of the MMW value and the LMW value increases or decreases by the presence or absence of occult blood. Thus, it is difficult to accurately evaluate the adiponectin level contained in the saliva, being affected by the presence or absence of occult blood.

On the other hand, in the present invention, the salivary adiponectin level is evaluated by the level of salivary adiponectin of 280 kDa or more, thereby reducing the effect of occult blood reaction, to make it possible to more accurately evaluate the adiponeetin level.

As a pathophysiological significance of evaluating high molecular weight (HMW) adiponectin contained in the saliva, usefulness as an acute non-invasive marker, a healthy marker in the mouth, and a temporal marker during following in a check station can be expected.

(2) As a method for evaluating a salivary adiponectin level, adiponectin may be evaluated using the quantitative value obtained by quantifying the reaction intensity when reacting an anti-adiponectin antibody which reacts specifically with multimeric adiponectin of 280 kDa or more contained in a saliva sample.

The intensity of the reaction with an antibody which reacts specifically with the multimeric adiponectin of 280 kDa or more is quantified and evaluated, whereby it is possible to evaluate the salivary adiponectin level further more without being affected by human multimeric adiponectin of less than 280 kDa (MMW and LMW) much present in the blood.

Here, the “anti-adiponectin antibody which reacts specifically with the multimeric adiponectin of 280 kDa or more” means an anti-adiponectin antibody in which the reaction with the multimeric adiponectin of 280 kDa or more is markedly intensive, as compared to the reaction with the multimeric adiponectin of less than 280 kDa. For example, the reaction intensity with the multimeric adiponectin of 280 kDa or more and the reaction intensity with the multimeric adiponectin of less than 280 kDa are quantified and compared, and when the former quantitative value is 10 times or more the latter quantitative value, it falls under the “anti-adiponectin antibody which reacts specifically with the multimeric adiponectin of 280 kDa or more” (here, a numerical expression of “the reaction intensity with the multimeric adiponectin of 280 kDa or more”/“the reaction intensity with the multimeric adiponectin of less than 280 kDa”≧10 is established).

Based on the experiment of the present inventor, it is confirmed that, for example, an isotype IgG1 antibody falls under the “anti-adiponectin antibody which reacts specifically with the multimeric adiponectin of 280 kDa or more”.

(3) The saliva sample is preferably a saliva sample collected by a straw.

Stress in the mouth can be suppressed by collecting the saliva sample by a straw, as compared to Salivette collection and gauze collection, and it is possible to evaluate the salivary adiponectin level further more accurately without being affected by occult blood.

(4) Also, in the evaluation of the salivary multimeric adiponectin level, the salivary adiponectin level may be evaluated by reacting saliva with a predetermined anti-adiponectin antibody, quantifying the reaction intensity of the predetermined anti-adiponectin antibody by the multimeric adiponectin of 280 kDa or more (for example, BMW reaction value), and also quantifying the reaction intensity of the predetermined anti-adiponectin antibody by the multimeric adiponectin of less than 280 kDa (for example, MMW and LMW reaction values), and obtaining the ratio of each quantified value (for example, HMW reaction value/MMW and LMW reaction values).

Alternatively, the salivary adiponectin level may be evaluated by reacting saliva with a predetermined anti-adiponectin antibody, quantifying the reaction intensity of the predetermined anti-adiponectin antibody by the multimeric adiponectin of 280 kDa or more (for example, HMW reaction value), and also quantifying the reaction intensity of the predetermined anti-adiponectin antibody by the multimeric adiponectin of all the molecular weights contained in the saliva (for example, HMW, MMW and LMW reaction values), and obtaining the ratio of each quantified value (for example, HMW reaction value/HMW, MMW and LMW reaction values).

The value (ratio) of [“HMW reaction value”/“MMW and LMW reaction values”], or the value (ratio) of [“HMW reaction value”/“HMW, MMW and LMW reaction values”] is obtained, whereby the proportion of the multimeric adiponectin of 280 kDa or more contained in the saliva can be obtained. The proportion of the multimeric adiponectin of 280 kDa or more is evaluated as the salivary adiponectin level, whereby it is possible to evaluate accurate adiponectin level with reduced effect of occult blood. Here, the reaction intensity of the high molecular weight adiponectin of 280 kDa or more or the reaction intensity of the high molecular weight adiponectin of less than 280 kDa can be obtained by measuring by Western Blotting method under non-denaturing and non-reducing conditions.

(5) Also, as the anti-adiponectin antibody used in the method for evaluating an adiponectin level,
when a saliva sample is fractionated into human multimeric adiponectin of 280 kDa or more and human multimeric adiponectin of less than 280 kDa, by Western Blotting method under non-denaturing conditions, to extract the band areas exceeding the upper and lower limits of 280 kDa, and reacted with a specific anti-adiponectin antibody, and a HMW quantitative value obtained by quantifying the reaction intensity in the band area of 280 kDa or more, and MMW/LMW quantitative values obtained by quantifying the reaction intensity in each of the band areas of less than 280 kDa are respectively calculated,
an anti-adiponectin antibody in which the HMW quantitative value is a value that can be significantly evaluated, and the MMW/LMW quantitative values are 1/10 or less of the HMW quantitative value or 0, may be selected and
the salivary adiponectin level may be evaluated by the reaction intensity of the selected anti-adiponectin antibody.

In the above method, an antibody that has the quantitative value of the band area of 280 kDa or more and does not have the quantitative value of the band area of less than 280 kDa or has only a small amount as compared to the quantitative value of the band area of 280 kDa or more is selected as the anti-adiponectin antibody. The salivary adiponectin level is evaluated by the significant reaction intensity of the human multimeric adiponectin of 280 kDa or more, by the selected anti-adiponectin antibody. Here, the “value that can be significantly evaluated” refers to an effective value that can be evaluated as reasonable reaction intensity, exceeding a measurement error when quantifying the reaction intensity by a plurality of measurements. For example, when it is a value of the predetermined minimum value or more in a plurality of samples, regardless of the difference in the used sample, and the maximum value of measurement error does not exceed the predetermined minimum value, the value is defined as the “value that can be significantly evaluated”.

In addition, a band image having the band areas each exceeding the upper and lower limits of 280 kDa is obtained by Western Blotting method under non-denaturing and non-reducing conditions, whereby, based on this band image, in each anti-adiponectin antibody of each saliva sample, the reaction intensity of high molecular weight (HMW) adiponectin of 280 kDa or more, medium molecular weight (MMW) adiponectin and low molecular weight (LMW) adiponectin of less than 280 kDa can be recognized as quantitative value. This expresses to which molecular weight area of adiponectin each and-adiponectin antibody has intensive reaction, and the hand image is extracted for a plurality of kinds of anti-adiponectin antibodies, whereby it is possible to select a preferable anti-adiponectin antibody of high molecular weight (HMW) adiponectin of 280 kDa or more.

More specifically, as a method for selecting an anti-adiponectin antibody for specifying the level of multimeric adiponectin of 280 kDa or more, it is preferred to select an anti-adiponectin antibody having: reactivity such that, the sample is fractionated into multimeric adiponectin of 280 kDa or more and oligomeric adiponectin of less than 280 kDa, by Western Blotting method under non-denaturing conditions, thereby extracting a molecular weight band image having the band areas exceeding the upper and lower limits of 280 kDa, to obtain the band level of 280 kDa or more and the band level of less than 280 kDa as a quantitative value, respectively, and the quantitative value of the band level of 280 kDa or more is large, and the quantitative value of the band level of less than 280 kDa can be regarded as small or substantially 0. For example, it is preferred to select an anti-adiponectin antibody such that the band level of less than 280 kDa is in a quantitative value ratio of at least less than 1, and preferably 1/10 or less, to the band level of 280 kDa or more.

(6) Also, a kit for measuring a salivary adiponectin level of this invention is a measurement kit for evaluating a salivary adiponectin level, comprising a reagent of an anti-adiponectin antibody which reacts specifically with multimeric adiponectin of 280 kDa or more in the collected saliva, wherein the salivary adiponectin level is evaluated by the level of the multimeric adiponectin of 280 kDa or more, by the reagent.

### Effects of the Invention

This invention has a constitution as described above, and evaluates an adiponectin level using a level of HMW adiponectin of 280 kDa or more, whereby it is possible to provide a method for evaluating a salivary adiponectin level with reduced effect of occult blood.

### BRIEF DESCRIPTION OF THE DRAWINGS

FIG. 1 shows distribution examples of multimeric adiponectin of a blood sample by Western Blotting method under non-denaturing conditions (first quantification method).

FIG. 2 shows quantification examples of a saliva sample by Western Blotting method under non-denaturing conditions (first quantification method).

FIG. 3 shows band image examples of a saliva sample by Western Blotting method under non-denaturing conditions (upper) and denaturing conditions (lower).

FIG. 4A shows band image examples of a saliva sample by Western Blotting method under non-denaturing conditions (upper) and denaturing conditions (lower).

FIG. 4B shows examples of profile plots based on the band images of Lanes 3 and 4 in FIG. 4A.

FIG. 4C shows examples of profile plots based on the band images of Lanes 10 and 11 in FIG. 4A.

FIG. 4D shows examples of quantitative values by profile plots based on the band images of Lanes 3 to 7 in FIG. 4A.

FIG. 5A shows measurement examples of multimeric adiponectin level excluding HMW, in each saliva sample of non-occult blood group and occult blood group.

FIG. 5B shows measurement examples of multimeric adiponectin level of HMW, in each saliva sample of non-occult blood group and occult blood group.

FIG. 6A shows band image examples of anti-adiponectin antibody 1 by Western Blotting method (first quantification method) under non-denaturing conditions.

FIG. 6B shows band image examples of anti-adiponectin antibody 2 by Western Blotting method (first quantification method) under non-denaturing conditions.

FIG. 6C shows band image examples of anti-adiponectin antibody 3 by Western Blotting method (first quantification method) under non-denaturing conditions.

FIG. 6D shows band image examples of anti-adiponectin antibody 4 by Western Blotting method (first quantification method) under non-denaturing conditions.

FIG. 7A shows measurement (quantification) examples of emission intensity of salivary adiponectin concentration by second quantification method, in each saliva sample of non-occult blood group and occult blood group.

FIG. 7B shows measurement (quantification) graph examples of emission intensity of salivary adiponectin concentration by second quantification method, in each saliva sample of non-occult blood group and occult blood group.

FIG. 8A shows band image examples by Western Blotting method (first quantification method) under non-denaturing conditions, in each sample or each collection method.

FIG. 8B shows examples of a profile plot based on each band image of FIG. 8A.

FIG. 9A shows measurement examples of BW values before and after dosing a soy genistein extract (GCP).

FIG. 9B shows measurement examples of BMI values before and after dosing a soy genistein extract (GCP).

FIG. 9C shows measurement examples of HMW adiponectin levels before and after dosing a soy genistein extract (GCP).

### EMBODIMENTS OF THE INVENTION

Hereinafter, an embodiment of this invention will be described.

### [Definitions of HMW, MMW, LMW]

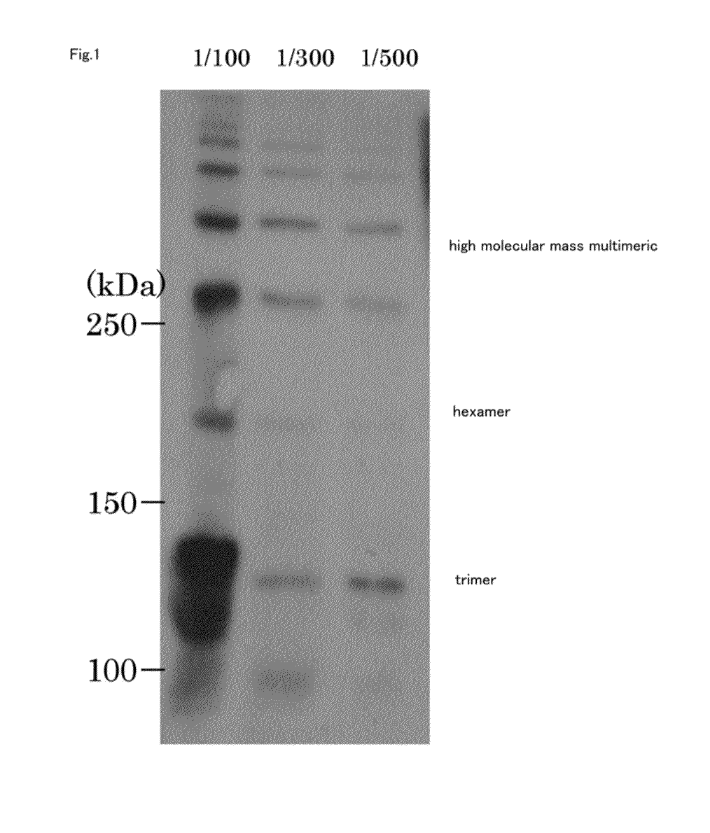

Adiponectin is an insulin sensitive hormone specifically secreted from fat cells, and is present in the blood in a relatively high concentration (5 to 10 μg/mL). Human multimeric adiponectin coexists in different multimeric structures like trimers, hexamers, dodecamers, or higher-order multimers in blood (refer to FIG. 1). These can be distinguished into three sections of high molecular weight (280 kDa or more, mainly around 300 kDa), medium molecular weight (150 kDa or more and less than 280 kDa, mainly around 160 kDa), and low molecular weight (0 kDa or more, and less than 150 kDa). Among them, those formed in high molecular weight region of 280 kDa or more are written as multimeric adiponectin or HMW in the present invention. Here, this multimeric adiponectin consists of dodecamers, octadecamers, or higher-order multimers. Also, medium molecular weight adiponectin consists of hexamers, and low molecular weight adiponectin consists of trimers.

A human having a mutation that cannot specifically form multimeric adiponectin tends to become diabetic, and further, high molecular weight adiponectin is particularly lowered in obesity and insulin resistance. Multimeric adiponectin rate is useful in the prediction of insulin resistance, as compared to the total adiponectin.

### (Conventional Measurement Method)

Examples of conventional method for measuring adiponectin include a method of subjecting a measurement sample to sodium dodecyl sulfite (SDS) denaturation treatment or heat denaturation treatment, followed by immunologically measuring adiponectin. This method is a method of exposing a conformationally hidden recognition site of antibody by the above treatment to immunologically measure the total level of adiponectin thus various multimers cannot be separately measured.

### [Effect of Occult Blood on Salivary Adiponectin Measurement]

Saliva was collected in gauze from 85 subjects using the Saxon original method at a health check station, and after being subjected to a centrifugal separation treatment, the saliva was extracted and cryopreserved. Salivary adiponectin level in each collected sample was measured by Alpha LISA (registered trademark) method of PerkinElmer Co., Ltd. The total average of the measured values of the salivary adiponectin level was 6.64 ng/ml on average. Also, in order to confirm the effect of occult blood reaction on the salivary adiponectin level measurement, the presence or absence of occult blood reaction by occult blood reaction test paper was examined, in each collected sample, and the collected samples were classified into an occult blood positive group and an occult blood negative group.

The average values of the salivary adiponectin levels by the AlphaLISA (registered trademark) method of PerkinElmer Co., Ltd. of the occult blood positive group and the occult blood negative group are respectively shown below.
Occult blood positive (33 subjects): 9.36 ng/ml on average
Occult blood negative (52 subjects): 4.91 ng/ml on average

Compared by the presence or absence of occult blood, remarkable difference, in that 9.36 ng/ml on average was determined for occult blood positive samples and 4.91 ng/ml on average was determined for occult blood negative samples, was recognized. This shows that the effect of having occult blood in the saliva is large, when measuring the adiponectin level by the AlphaLISA (registered trademark) method. As described above, in the AlphaLISA (registered trademark) method, it is sometimes difficult to accurately evaluate the salivary adiponectin level, being affected by the presence or absence of occult blood depending on the used antibody.

### (First Quantification Method of the Present Invention)

In contrast to the above conventional method, as the first quantification method of the present invention, the molecular weight distribution of human multimeric adiponectin in a blood sample and a saliva sample was evaluated by Western Blotting method. Specifically, the collected sample was separated by SDS-PAGE electrophoresis by polyacrylamide (3 to 8%), transferred to a PVDF membrane, and reacted with a monoclonal antibody, and the band image showing reaction intensity was detected (Western Blotting method, FIGS. 1 to 4).

### (Molecular Weight Distribution in Blood Sample)

The band image examples of blood, samples by Western Blotting method under non-denaturing and non-reducing conditions are shown in FIG. 1. Adiponectin is present in the blood in high concentration, thus the blood samples were each diluted to 1/100, 1/300, or 1/500, and compared and studied. MAB3604 of Millipore Corporation that is a mouse IgG1 antibody was used as an anti-adiponectin antibody.

According to FIG. 1, as the molecular weight distribution of adiponectin in the blood, the low molecular weight adiponectin of 100 to 150 kDa is highest, and subsequently, the medium molecular weight adiponectin of 150 to 280 kDa, and the high molecular weight adiponectin of 280 kDa or more are distributed in this order. Comparing the distribution amount of each region of upper and lower than 280 kDa as a threshold, it can be said that MMW and LMW adiponectin of less than 280 kDa are present more than HMW of 280 kDa or more in the blood (Refer to FIG. 1).

### (Molecular Weight Distribution in Saliva Sample)

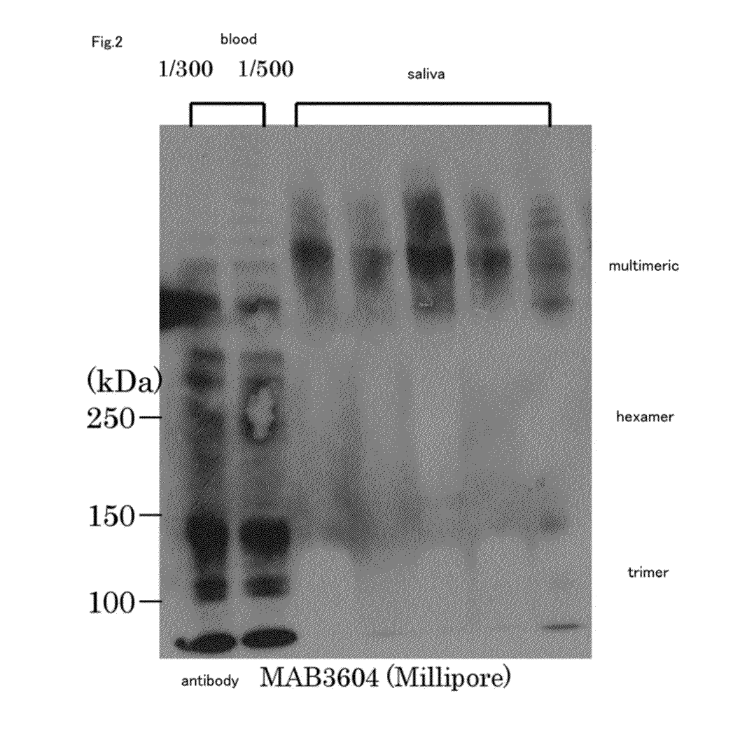

The band image examples of the saliva, samples by Western Blotting method under non-denaturing and non-reducing conditions are shown in FIG. 2 (right five lanes in FIG. 2). Then for comparison, the band image examples of the blood samples are also shown in left two lanes in FIG. 2. In the saliva samples (right five lanes in FIG. 2), the HMW of 280 kDa or more is significantly strongly expressed, and the MMW and LMW of less than 280 kDa are only less expressed compared to the HMW. Moreover, in the blood sample (left two lanes in FIG. 2), the MMW and LMW of less than 280 kDa are expressed in the same level as or stronger than the HMW of 280 kDa or more. Based on this, it can be said that, when the molecular weight distribution of adiponectin in the saliva sample is distinguished into each region of upper and lower than 280 kDa as a threshold, and the molecular weight distribution of human multimeric adiponectin is compared, in the molecular weight distribution in the saliva, the HMW of 280 kDa or more is present more than the MMW and LMW of less than 280 kDa, unlike the molecular weight distribution in the blood.

The anti-adiponectin antibody used in the band images shown in FIG. 2 is MAB3604 of Millipore Corporation that is a mouse IgG1 antibody. When using this antibody, it is confirmed that different reactions are shown between the blood and the saliva. More specifically, in the band images of left two lanes in FIG. 2 (1/300 diluted sample of the blood, 1/500 diluted sample of the blood), the medium molecular weight adiponectin and the low molecular weight adiponectin are strongly reacted, and the multimeric adiponectin is hardly reacted. Also, in any band images of right five lanes in FIG. 2 (five undiluted saliva samples), the multimeric adiponectin is all strongly reacted, and the medium molecular weight adiponectin and the low molecular weight adiponectin are hardly reacted.

More specifically, when this antibody is reacted with the saliva sample, to form band images of the multimeric adiponectin by the molecular weight, and the band level of 280 kDa or more and the band level of less than 280 kDa are each quantified, the quantitative value of the HMW of 280 kDa or more is significantly large, and the quantitative values of the MMW and LMW of less than 280 kDa are smaller than the HMW, or the MMW and LMW are not substantially contained.

Based on the above, it can be said that MAB3604 of Millipore Corporation is an antibody that specifically reacts with the multimeric adiponectin of 280 kDa or more in the saliva sample. When adiponectin is reacted with this antibody and evaluated using the quantitative value obtained by quantifying the intensity of the reaction, the salivary adiponectin level can be obtained by specifying the intensity of the reaction of the multimeric adiponectin, with suppressing the effect of blood contamination into the saliva.

As the method for quantifying the reaction intensity of the band images, the band images are profile plotted by image analysis, and the enclosed area is measured from the zero value containing the peak part, thereby digitizing the area of each peak contained in the plot of the band images. As a comparative example, standard synthetic adiponectin in which actual mixed amount is detected can be digitized by band imaging in the same conditions. By the comparison of these numerical values, the numerical value of plot of the band images in the saliva can be converted into the actual level.

### (Comparison Under Denaturing Conditions and Non-denaturing Conditions)

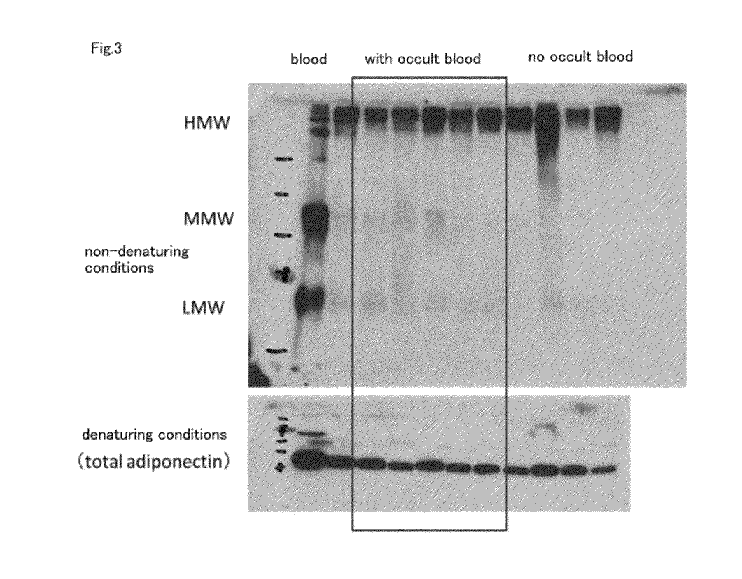

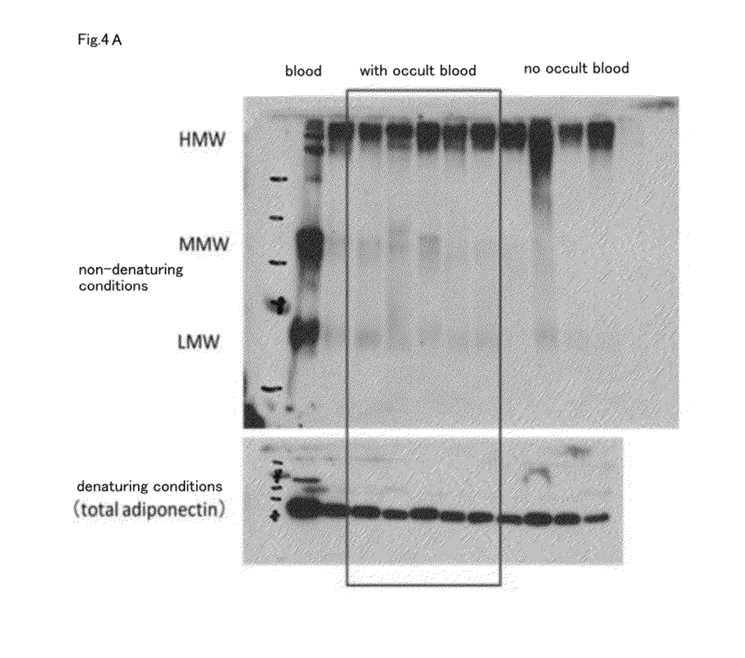

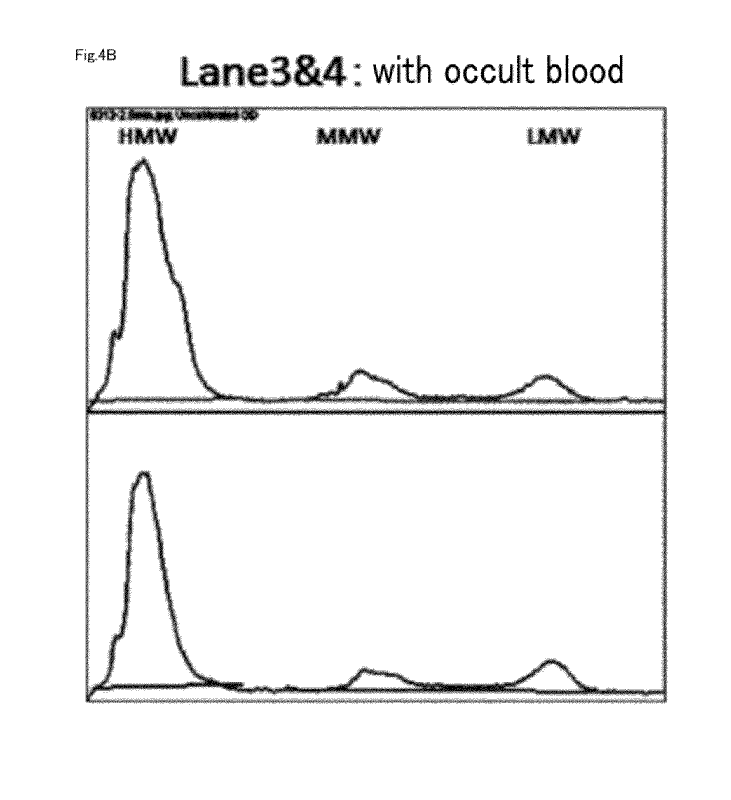

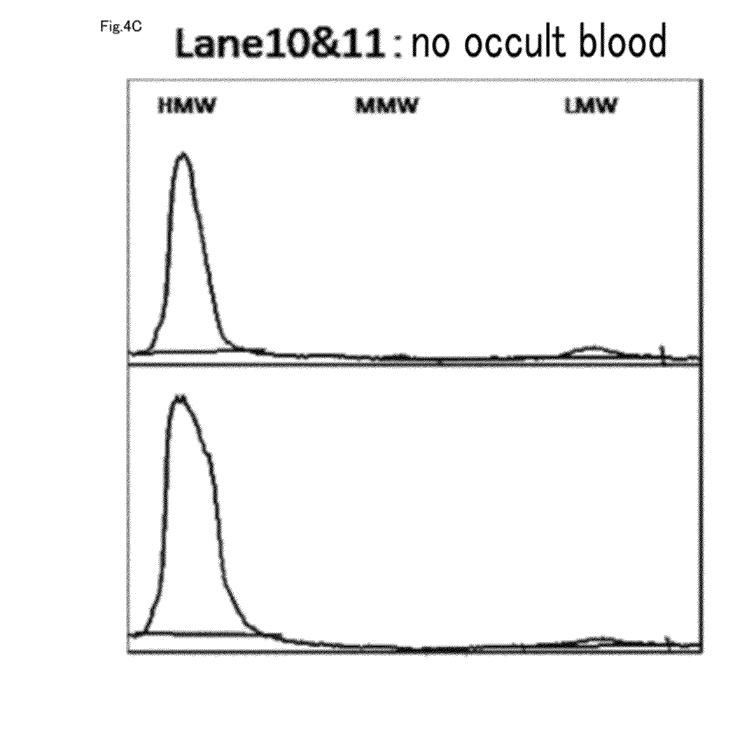

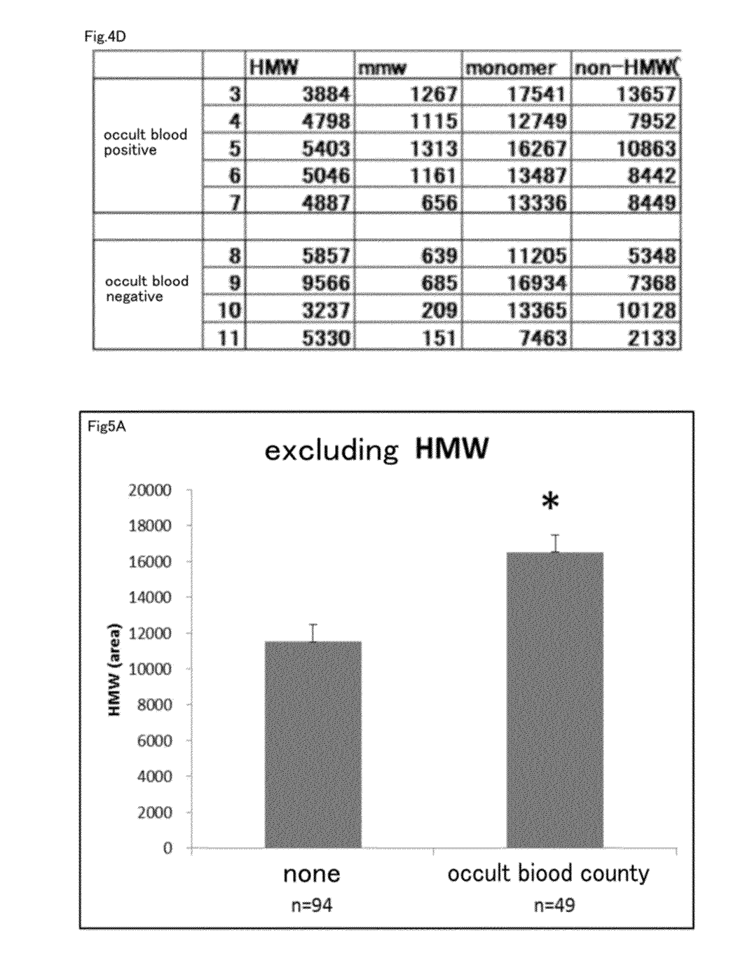

For each of blood sample, saliva sample with occult blood in which the blood is contaminated, and saliva sample without occult blood, band images by Western Blotting method under non-denaturing conditions (FIG. 3 upper figure, FIG. 4A upper figure) and band images by Western Blotting method under denaturing conditions (FIG. 3 lower figure, FIG. 4A lower figure) were formed. As the denaturing conditions, 2-mercaptoethanol was mixed, and the mixture was heated at 95° C. for 10 minutes. The anti-adiponectin antibody used in the band images shown in FIGS. 3 and 4 is MAB3604 of Millipore Corporation, that is a mouse IgG1 antibody. In this antibody, medium molecular weight and low molecular weight adiponectin are much reacted in the blood sample, and the reaction of these medium molecular weight and low molecular weight adiponectin is markedly expressed even under denaturing conditions.

Each lane of the obtained band images in FIG. 4A was extracted and the band concentrations of each lane were profile plotted with a molecular weight as the horizontal axis, and quantified by image analysis. In this image analysis, the profile plot images of the third and fourth lanes from the left in FIG. 4A are shown in FIG. 4B. Also, the profile plot images of the tenth and eleventh lanes from the left in FIG. 4A are shown in FIG. 4C. The area enclosed by the peak part of each profile plot and the baseline is measured, whereby each multimeric adiponectin level of HMW, MMW and LMW can be digitized, namely, quantified. The data table of the quantitative values by the same profile plot images as in FIGS. 4B and 4C is shown in FIG. 4D. The numbers in the table are a quantitative value of HMW, a quantitative value of MMW a quantitative value of the number of monomers, and a quantitative value of only MMW and LMW excluding HMW, respectively, from the left.

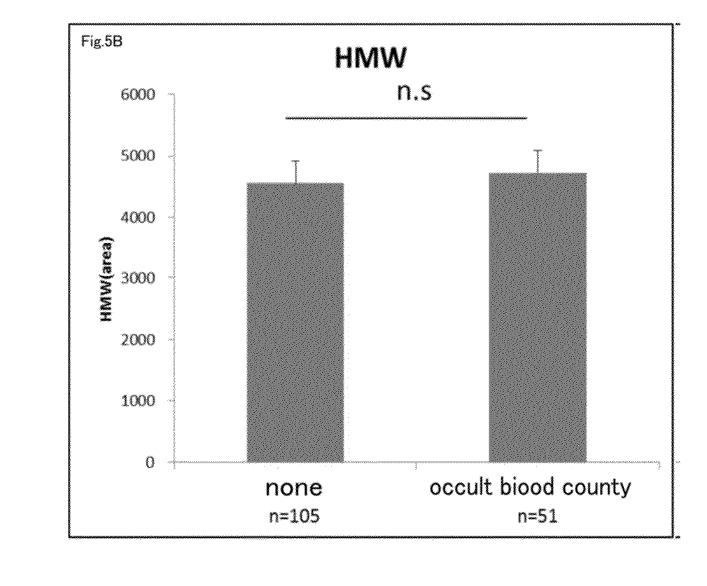

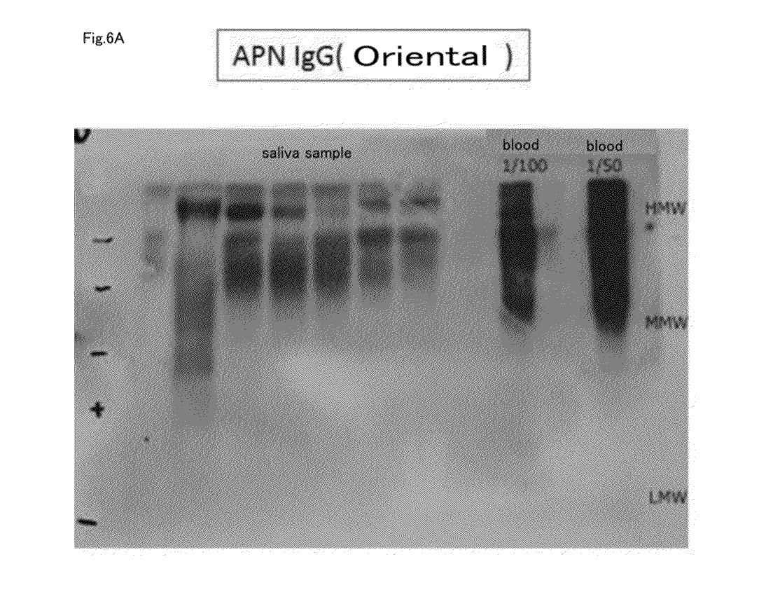

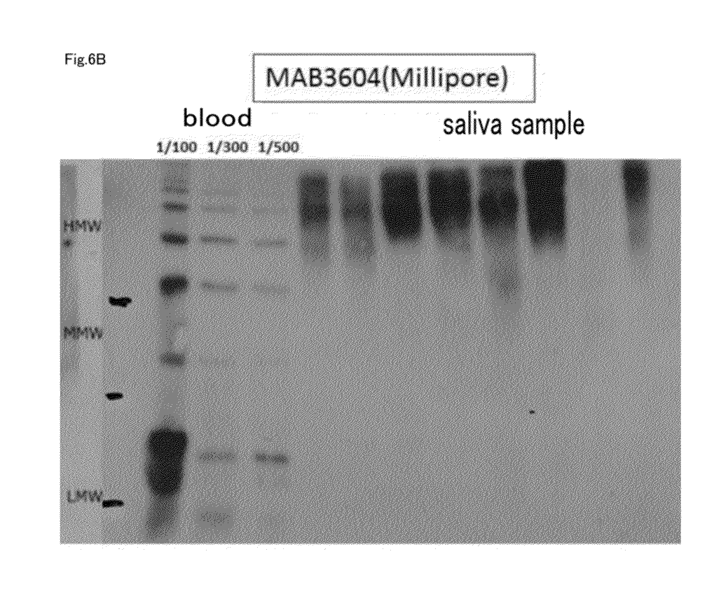

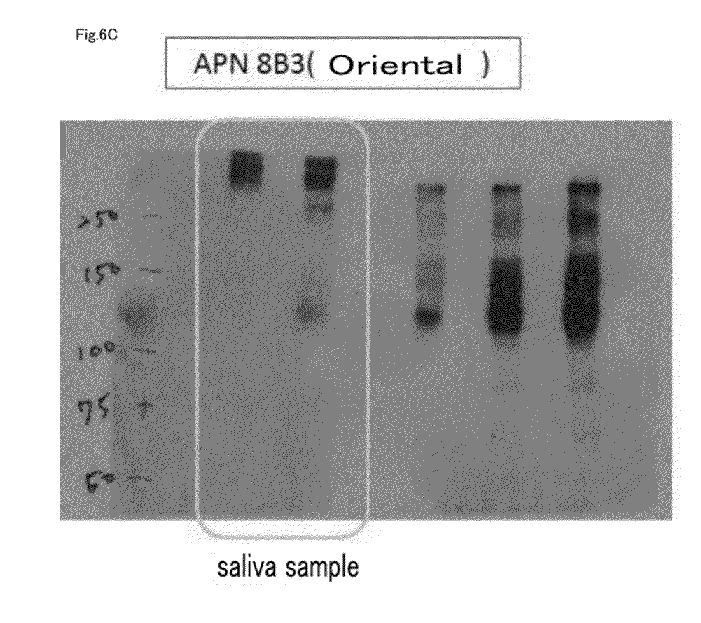

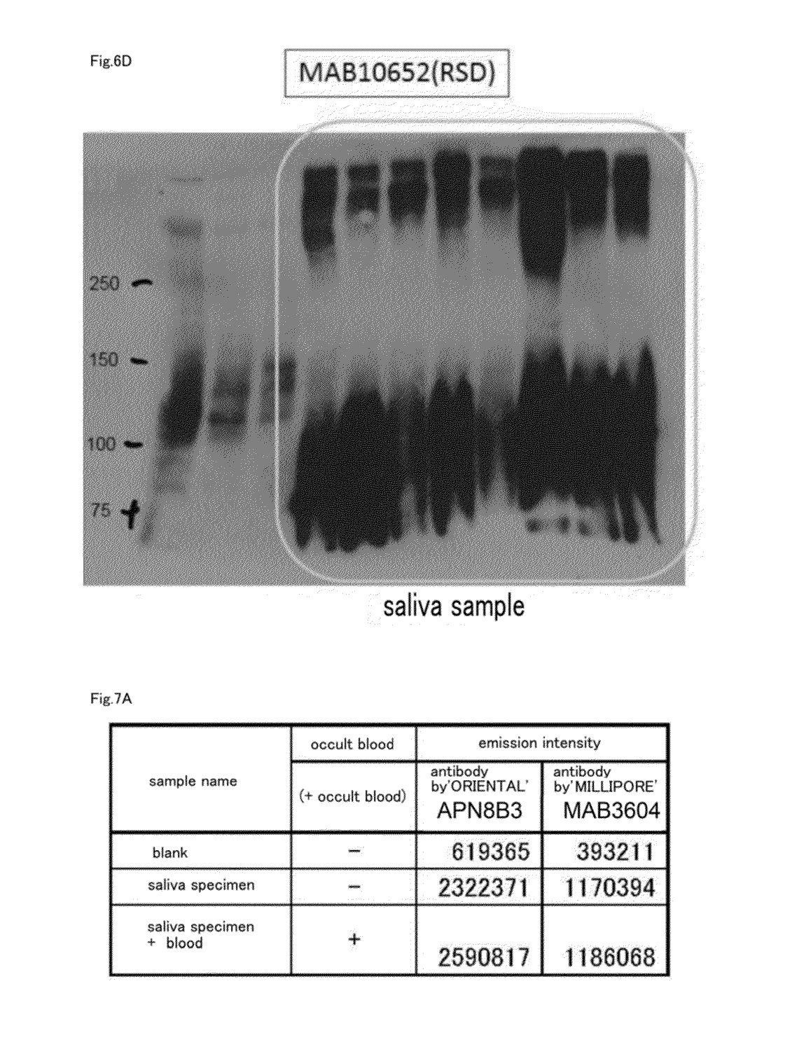

The saliva sample used in Western Blotting method under denaturing conditions described above was fractionated by Western Blotting method under non-denaturing conditions, and the band concentration was binalized along with the band area with 280 kDa as the upper and lower limits and quantified by image analysis. The obtained quantitative value of the band concentration of 280 kDa or more was non-significant, regardless of the presence or absence of occult blood (FIG. 5B).

In addition, the quantitative value of adiponectin of less than 280 kDa and MMW) calculated by subtracting the quantitative value obtained by quantifying the adiponectin level of 280 kDa or more from the quantitative value obtained by quantifying the total salivary adiponectin level is shown in FIG. 5A. As compared to the quantitative value of HMW that gives no significant difference by the presence or absence of occult blood, the quantitative values of LMW and MMW give a significant difference by the presence or absence of occult blood.

The adiponectin level of the saliva is evaluated using the adiponectin multimers of 280 kDa or more not affected by the presence or absence of occult blood, whereby it is possible to accurately evaluate adiponectin not affected by occult blood.

### (Method for Selecting Anti-adiponectin Antibody)

As a preferred method for selecting an antibody for evaluating salivary adiponectin using the level of multimeric adiponectin of 280 kDa or more as described above, a quantification method comprising fractionating using a predetermined anti-adiponectin antibody by Western Blotting method under non-denaturing conditions, extracting only the molecular weight bands of the upper and lower limits of 280 kDa, and quantifying the band images of the molecular weight bands of the upper and lower limits of 280 kDa by profile plotting a graph of the molecular weight distribution of the image concentration.

Specifically, the molecular weight distribution of adiponectin multimers in the saliva sample is obtained by using Western Blotting method under non-denaturing conditions. Staining is performed using a predetermined anti-adiponectin antibody in the obtained molecular weight distribution, the band areas of the upper and lower limits of 280 kDa are extracted, and quantified by profile plotting, a graph of the molecular weight distribution of the image concentration to obtain a quantitative value. Specifically, the molecular weight band images having the band areas exceeding the upper and lower limits of 280 kDa are extracted, and the band level of 280 kDa or more and the band level of less than 280 kDa are obtained as a quantitative value, respectively.

The quantitative value of the adiponectin multimers of 280 kDa or more and the quantitative value of the adiponectin multimers of less than 280 kDa obtained as above are compared, and an antibody is selected such that a ratio of the levels of MMW and LMW of less than 280 kDa to the level of HMW of 280 kDa or more is at least less than 1, preferably 1/10.

The conditions of the selection include whether the reactivity is such that the quantitative value of the band level of 280 kDa or more is large, and the quantitative value of the band level of less than 280 kDa is small or can be regarded as substantially 0. For example, an anti-adiponectin antibody can be selected such that the band level of less than 280 kDa is in a quantitative value ratio of at least less than 1, preferably in a ratio of 1/10 or less, to the band level of 280 kDa or more. The anti-adiponectin antibody satisfying the above conditions is selected, whereby it is possible to obtain an antibody having high reactivity to the level of the multimeric adiponectin of 280 kDa or more contained in the saliva. The above antibody is selected and used in evaluation of the adiponectin level, whereby it is hardly affected by adiponectin multimers of MMW or LMW of less than 280 kDa increased by the effect of occult blood, thus more accurate evaluation is possible.

### [Each Reaction Tendency of a Plurality of Antibodies]

For four kinds of antibodies, anti-adiponectin antibodies to suitable adiponectin multimers of 280 kDa or more were evaluated using the antibody selection method described above. As the four kinds of antibodies, APN IgG (rabbit IgG) manufactured by Oriental Yeast Co., Ltd., MAB3604 (mouse IgG1) manufactuted by Millipore Corporation, APN 8B3 (mouse IgG1K) manufactured by Oriental Yeast Co., Ltd., and MAB10652 (mouse IgG2B) manufactured by RSD were used.

The band images fractionated by Western Blotting method under non-denaturing conditions using the four kinds of antibodies are shown in FIG. 5A to FIG. 5D). As to the reaction tendency of the antibodies, in Reaction antibodies 2 and 3, the molecular weight part of adiponeetin of 280 kDa or more was markedly detected, and detection of the molecular weight part of low molecular weight adiponectin was small. On the other hand, in Reaction antibodies 1 and 4, the molecular weight part of low molecular weight adiponectin was markedly detected together with the molecular weight part of adiponectin of 280 kDa or more, and it could not be said that the molecular weight part of low molecular weight adiponectin was small.

In the four kinds of antibodies, the anti-adiponectin antibody suitable for the adiponectin multimers of 280 kDa is MAB3604 (mouse IgG1) manufactured by Millipore Corporation, and APN 8B3 (mouse IgG1K) manufactured by Oriental Yeast Co., Ltd. Both are an isotype IgG1 antibody (mouse IgG1), and it can be said that at least IgG1 antibody is preferred for specification and evaluation of the salivary HMW level.

### (Second Quantification Method)

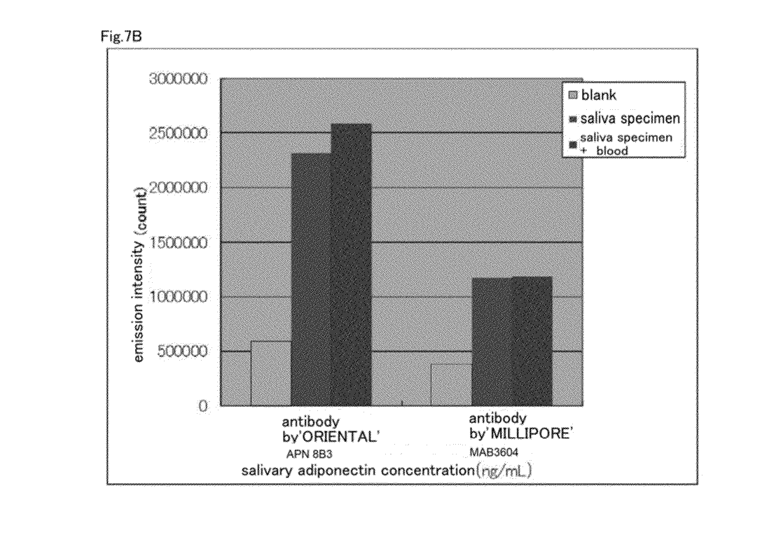

As a second quantification method other than Western Blotting method, a procedure using an antigen-antibody reaction represented by ELISA method can be used. The present inventors, as the second quantification method, added a saliva sample or a occult blood sample obtained by contaminating the blood into the saliva sample dropwise to each well of a microwell, and added MAB3604 (mouse IgG1) manufactured by Millipore Corporation diluted 5,000 times, and APN 8B3 (mouse IgG1K) manufactured by Oriental Yeast Co., Ltd., as a primary antibody, then added a secondary antibody modified with an emission substrate, and measured an emission intensity by a fluorescent plate reader. The results are shown in FIG. 7A and FIG. 7B.

According to the middle line in FIG. 7A, the emission intensity of the saliva specimen of APN 8B3 (the same as Antibody 3) of Oriental Yeast Co., Ltd. was 2322371, and significant difference was not seen, as compared to an emission intensity of the blood contamination specimen of the same antibody of 2590817. Also, according to the right line in FIG. 7A, the emission intensity of the saliva specimen of MAB3604 antibody (the same as Antibody 2) of Millipore Corporation was 1170394, and significant difference was not seen, as compared to an emission intensity of the blood contamination specimen of the same antibody of 1186068. Even seeing the right and middle graphs of three line graph in FIG. 7B, when comparing the emission intensities of the occult blood sample into which the blood is contaminated and the saliva sample into which the blood is not contaminated, significant difference cannot be obtained. Based on this, it can be said that it is possible to more accurately evaluate the adiponectin multimers of 280 kDa or more, without being affected by the presence or absence of blood contamination (occult blood).

Other than the above, the salivary adiponectin level can be quantified, using the various methods of extracting, measuring or quantifying the adiponectin level, such as latex method, immunochromatographic method, turbidimetric method, nephelometric method, indirect immunofluorescence method (IIF), CLEIA method (chemiluminescence enzyme immunoassay method), ECLIA method (electrochemical luminescence immunoassay method), and FPIA method (fluorescence polarization immunoassay assay). In any method, the salivary HMW level can be more accurately specified, by using an anti-adiponectin antibody showing a reaction specific to the adiponectin multimers of 280 kDa (for example, antibody of mouse IgG1).

### [Effect of Difference in Collection Method]

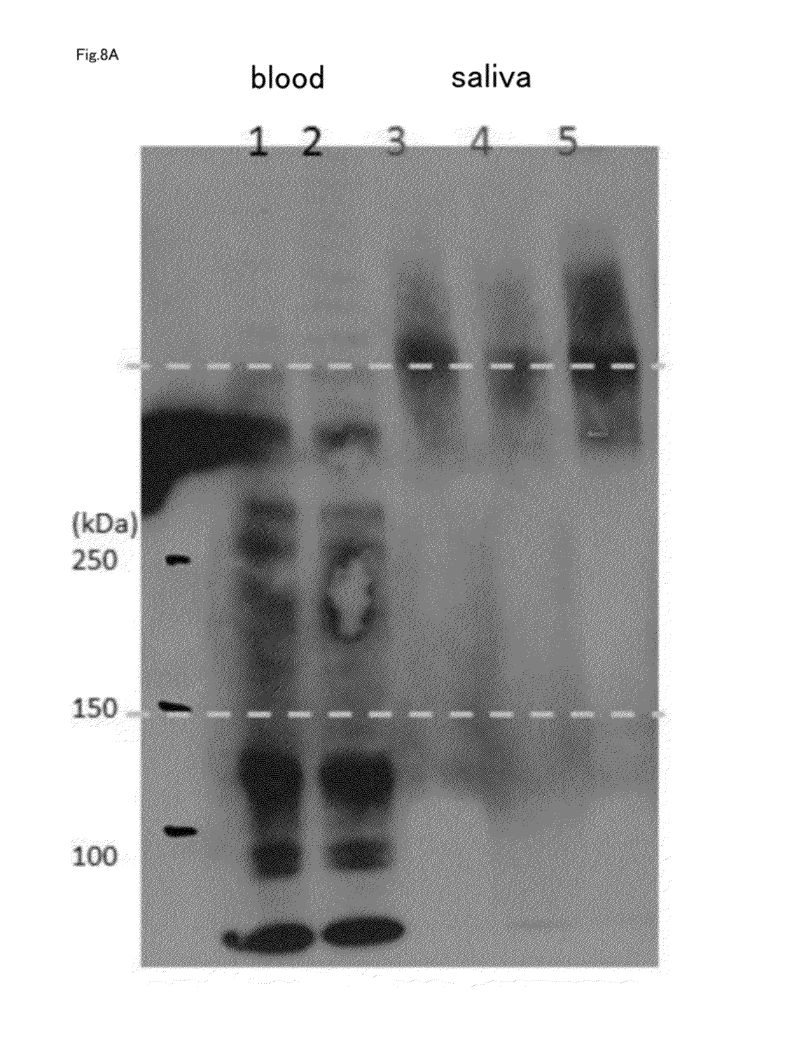

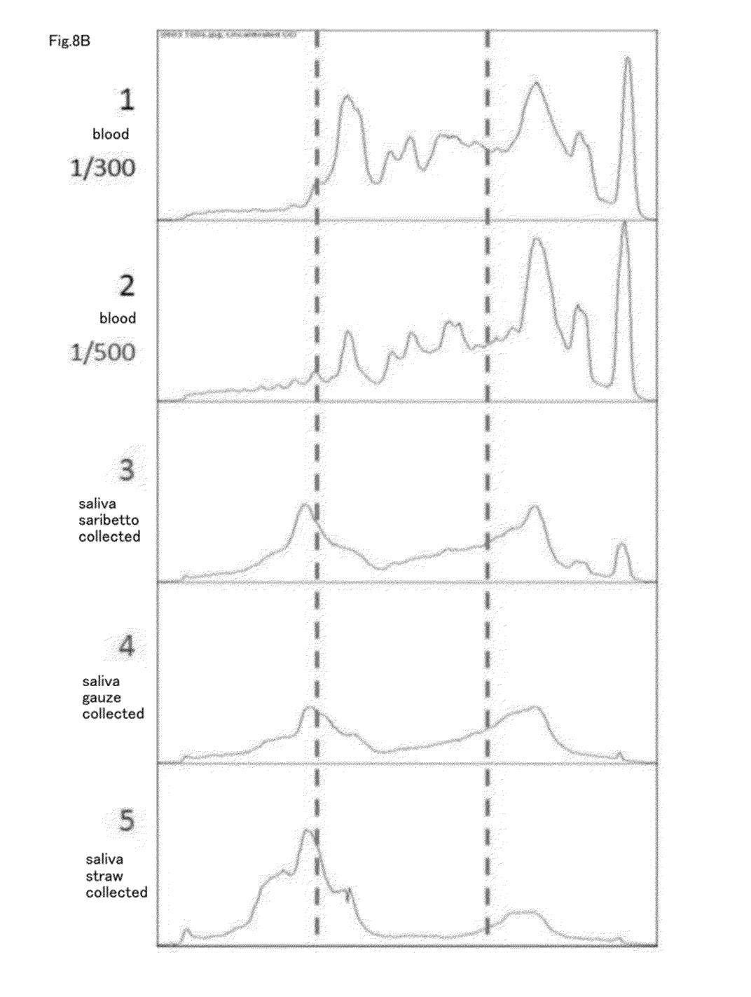

When the effect of difference in the collection method as shown in FIG. 8 is studied, it was found to be preferred that the saliva sample is a saliva sample obtained by directly collecting a secretion from secretory gland by a straw.

The present inventor collected a human saliva sample, respectively by three types of collection methods comprising Salivette collection, gauze collection and straw collection, and fractionation was performed using the saliva sample by each collection method by Western Blotting method under non-denaturing conditions. Also, as comparative samples, 300-fold diluted and 500-fold diluted blood samples were also fractionated in the same way. The results are shown in FIG. 8A and FIG. 8B. FIG. 8A shows band images of each sample by Western Blotting method under non-denaturing conditions. Graphs of a profile plot of the band concentration of each band area in the molecular weight distribution axis are shown in FIG. 8B. The lowest profile plot graph in FIG. 8B shows a molecular weight distribution of the intensity of detection reaction of the saliva sample by straw collection. Compared to other graphs, it can be determined that, in the saliva sample by straw collection, the detection reaction of HMW of 280 kDa or more is largest, and the detection reactions of MMW and LMW of less than 280 kDa are smallest. Also, it can be determined that the detection reactions of MMW and LMW are half or less of the detection reaction of HMW.

In FIG. 8B, each of profile plot graphs of the thud from the bottom and the second from the bottom shows the molecular weight distribution of the intensity of the detection reaction of the saliva sample by Salivette collection and gauze collection, respectively. According to these graphs, it can be determined that, in the case of Salivette collection or gauze collection, the detection reaction of HMW is slightly weak as compared to the case of straw collection, and the detection reactions of MMW and LMW are almost equivalent to the detection reaction of HMW. The reason for this is assumed that, in a case where there is oral bleeding in Salivette collection or gauze collection, the oral cavity is stimulated when putting Salivate or gauze in the mouth and biting, it, thereby increasing the bleeding amount, and the molecular weight distribution changes when separating the saliva from the gauze or Salivette.

Based on the comparison of the collection method shown in FIG. 8A and FIG. 8B above, among the three types of collection methods comprising Salivette collection, gauze collection and straw collection, the collection method by straw collection least affected by contamination of multimeric adiponectin of less than 280 kDa was evaluated as directly reflecting the state of the raw saliva secreted from secretory gland. More specifically, it was found that, it is preferred to quantify the reaction intensity using an anti-adiponectin antibody reacting with the adiponectin multimers of 280 kDa or more, using the saliva sample by straw collection, and whereby the salivary adiponectin level can be more accurately evaluated with less effect of contamination of blood or the like.

Also, the salivary adiponectin level can be evaluated as a relative value, using the level of adiponectin of 280 kDa or more, the total adiponectin level or the level of adiponectin of less than 280 kDa. In this case, the saliva sample is convened into a quantitative value by the first quantification method and the second quantification method, and evaluated by the level of adiponectin of 280 kDa or more in the total adiponectin level.

Specifically, the level of adiponectin of 280 kDa or more is first quantified by the first quantification method. In the first quantification method, using Western Blotting method under non-denaturing conditions, the level of adiponectin of 280 kDa or more is quantified by obtaining the molecular weight distribution of the adiponectin multimers in the saliva sample, then extracting, the band area of 280 kDa or more, profile plotting the graph of the molecular weight distribution of image concentration, and calculating the area of the peak part of the graph. Also, the total salivary adiponectin level is quantified by the second quantification method. In the second quantification method, using Western Blotting, method under denaturing conditions, the total adiponectin level is quantified by extracting the hand area of the total adiponectin multimers in the saliva sample that is non-fractionation, profile plotting, the graph of the molecular weight distribution of image concentration, and calculating the area of the peak part of the graph.

In addition, the total adiponectin level may be quantified by obtaining the molecular weight distribution of the adiponectin multimers in the saliva sample using the first quantification method, without using the second quantification method, extracting all the band areas, profile plotting the graph of the molecular weight distribution of image concentration, and calculating the area of the peak part of the obtained graph. In addition, instead of the total adiponectin level, the level of salivary adiponectin of 280 kDa or more may be relatively evaluated using the adiponectin multimers of less than 280 kDa.

### (Kit for Measuring Salivary Adiponectin Level)

Also, the present invention is established as a kit for measuring a salivary adiponectin level, using the method for evaluating a salivary adiponectin level as described above. The measurement kit for evaluating a salivary adiponectin level of the present invention comprises at least a reagent of anti-adiponectin antibody specifically reacting with the multimeric adiponectin of 280 kDa or more in the saliva sample, and the salivary adiponectin level can be evaluated by the level of multimeric adiponectin of 280 kDa or more, by the reagent.

The measurement kit preferably comprises a collecting means having a straw-like pickup device for collecting a saliva sample, and a reaction plate of an anti-adiponectin antibody that specifically reacts with the multimeric adiponectin of 280 kDa or more in the saliva sample.

The collecting means for collecting a saliva sample comprises, for example, straw for collection and a storage container. The measurement kit containing a reagent of an anti-adiponectin antibody is constituted, for example as a measurement kit containing a reaction plate of an anti-adiponectin antibody. Here, the anti-adiponectin antibody specifically reacts with the multimeric adiponectin of 280 kDa or more in the saliva sample, and is an antibody selected by the above method for selecting an antibody. For example, an antibody of isotype IgG1 falls under the anti-adiponectin antibody.

It is possible to evaluate the salivary adiponectin with the level of multimeric adiponectin of 280 kDa or more, by the above measurement kit. When evaluating a salivary adiponectin level using the above measurement kit, it is constituted as the evaluation kit comprising a measuring means that measures the reaction intensity with the saliva sample by the anti-adiponectin antibody and quantifying means that quantifies the reaction intensity, in addition to the collecting means and reaction plate as described above.

Using each constitution of this measurement kit, the saliva sample collected by the collecting means is reacted with the anti-adiponectin antibody of the reaction plate. Also, using each constitution of the evaluation kit, the reaction intensity of the anti-adiponectin antibody with the saliva sample is measured and quantified by the measuring means, and the saliva adiponectin level is evaluated by the level of multimeric adiponectin of 280 kDa or more obtained by the quantification.

### [Non-invasive Evaluation by Intervention of Functional Food]

This time, non-invasive inspections (counseling, body composition measurement, saliva collection) were conducted with office workers once a month, and functional food suitable for individuals was selected by diet evaluation using a good diet sheet, and intervention was provided by doses for 3 months. Among 37 males provided with intervention for 3 months, for 8 males dosing a soy genistein extract (GCP), HMW adiponectin of the saliva was detected by Western Blotting method (first quantification method) under non-denaturing conditions. Soy genistein extract (GCP) contains isoflavone which is found to increase human multimeric adiponectin.

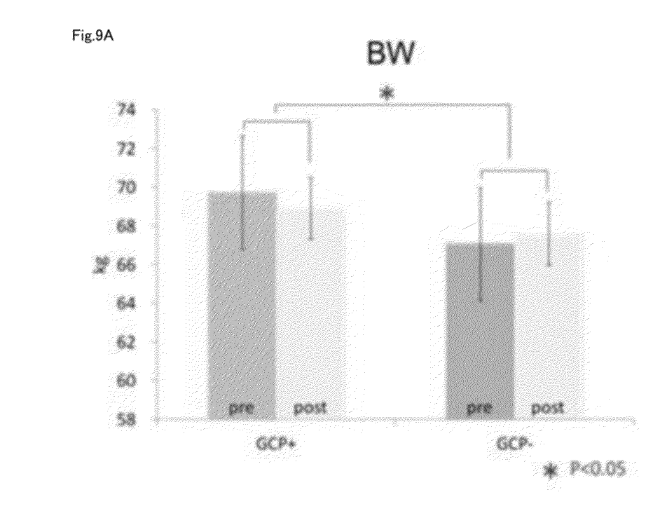

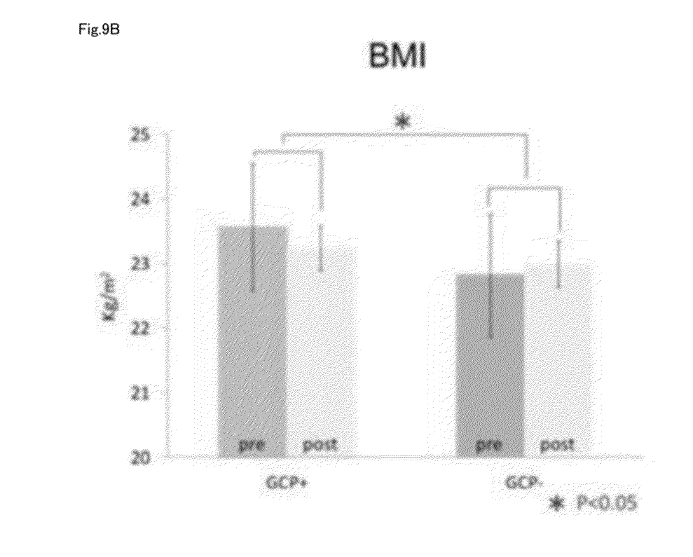

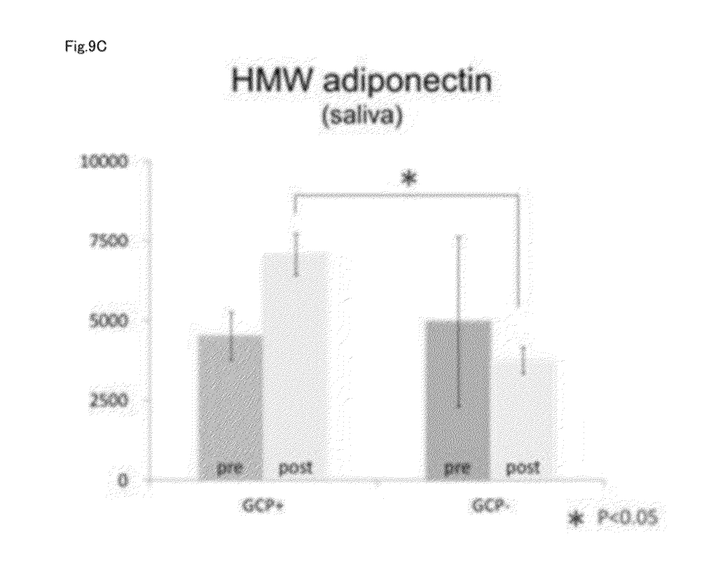

As a result, the BW (body weight) increase and BMI (body mass index) increase were significantly suppressed in a person who ingested GCP, as compared to a person who did not ingest GCP (FIG. 9A and FIG. 9B). Furthermore, in a person who ingested GCP, HMW adiponectin of 280 kDa or more significantly increased (FIG. 9C). Also, in adiponectin multimers of 280 kDa or more, body weight and BMI, correlation by GCP doses was obtained. Based on this, using the quantitative value of BMW as the salivary adiponectin level, the ingestion effect of GCP (effect of suppressing body weight increase, effect of suppressing BMI increase) can be evaluated. For example, when the quantitative value of adiponectin level with a gauge value of 5500 or more was obtained in FIG. 9C, it can be evaluated that the effect of does of this dosed article (GCP) is significant. In this intervention to the office workers for a short period of 3 months, it is suggested that the intervention of GCP doses by a good diet sheet and non-invasive evaluation is effective in lifestyle disease prevention, and also suggested that HMW adiponectin is an acute non-invasive marker before and after functional food administration or the like.

Also, in an adiponectin level of 4 μg/ml or more in the blood, it is considered that there is a possibility of low adiponectinemia, and metabolic syndrome. The judgment reference value of the salivary HMW adiponectin level, as well as the judgment reference value of 4 μg/ml in the blood, is set, and the risk of low adiponectinemia can be evaluated, based on whether the measured HMW quantitative value, is the judgment reference value, or more. In the setting of the judgment reference value in the saliva in this case, it is necessary to take correlation between the data of quantitative value of an antibody reaction using a specific anti-adiponectin antibody that specifically reacts with HMW adiponectin and the relevance to low adiponectinemia. The data by the predetermined quantification method using the specific anti-adiponectin antibody is accumulated, as this procedure, whereby a marker of symptoms correlated with the adiponectin level of a human or biological body is set, and each symptom can be evaluated.

### [Possibility of Oral Care]

The main theme of the World Health Day of the World Health Organization (WHO) in 1994 was “oral health that contributes to healthy life”. It is clear that the basis of healthy life is oral health. As the life is prolonged, the cause of losing teeth becomes periodontal disease. The American Dental Association has announced that as much as 80% of people of age 65 or more in America has periodontal disease. Further problem is that periodontal disease has an important association with diabetes, respiratory diseases, pregnancy trouble, and heart disease.

The prevention of periodontal disease is one of measures against metabolic syndrome. Periodontal disease is a factor having adverse effect on diabetes that is a cause of metabolic syndrome. Since periodontal disease is less likely to have subjective symptoms, bacteria are unnoticeably carried, from the teeth into the body. Today, the relationship between metabolic syndrome and periodontal disease has been pointed out. One playing an important action in the mechanism is a substance called cytokine. Cytokine is a protein produced by lymphocytes and the like, and works for maintaining homeostasis. However, when cytokine is excessively produced by bacterial infection and the like, it negatively affects on a living body. Antimicrobial proteins such as lysozyme, histatin and cystatin contained in the saliva suppress breeding and toxicity of bacteria and regulate the immune system to work for infection protection.

It is considered that the HMW adiponectin directly secreted from salivary gland and much contained in the saliva, as well as the HMW in the blood, is possibly useful as an oral healthy marker.

### [Possibility of Salivary Cell-derived Recombinant HMW Adiponectin Protein]

It is suggested that HMW secreted from fat cells is highly functional as compared to MMW and LMW, and when culture of human salivary gland cells becomes possible in the future, it is possible that the secreted HMW adiponectin becomes very useful recombinant protein.

The present evaluation method is described as a method for evaluating salivary adiponectin in each of the above examples, however, it is not used in medical practice or regeneration treatment action containing biomaterial, but used in scientific analysis or statistics except for medical practice or regeneration treatment action. In addition, each of the above evaluation methods can be applied as it is to the method for specifying a salivary adiponectin level, for the purpose of scientific analysis or statistics of the saliva. Furthermore, it can be applied as a measurement kit for analysis and evaluation of a salivary adiponectin level, using the equipment used in each evaluation method.

Moreover, human salivary multimeric adiponectin is specified or evaluated in each of the above examples, and it is not limited to the multimeric adiponectin level based on human, but a saliva sample of a biological body having salivary gland (animal bred as pet or livestock) is collected, whereby the salivary adiponectin level of a wide variety of biological bodies can be specified or evaluated.

### INDUSTRIAL APPLICATBILITY

A large scale and long term follow-up of the salivary adiponectin level is implemented, by providing a (non-invasive) method for evaluating adiponectin which is less affected by the presence or absence of occult blood, whereby further development is expected, naturally in lifestyle disease such as metabolic syndrome and dry mouth, and even M various health care fields including oral care.

1. A method for evaluating a salivary adiponectin level, comprising evaluating the salivary adiponectin level by the level of multimeric adiponectin of 280 kDa or more.

2. The method for evaluating a salivary adiponectin level according to claim 1, using the quantitative value obtained by quantifying the reaction intensity when reacting an antibody which reacts specifically with the adiponectin multimers of 280 kDa or more contained in a saliva sample.

3. The method for evaluating a salivary adiponectin level according to claim 1, wherein the saliva sample is a saliva sample collected by a straw.

4. The method for evaluating a salivary adiponectin level according to claim 1, comprising:
reacting saliva with a predetermined anti-adiponectin antibody,
quantifying the reaction intensity of the predetermined anti-adiponectin antibody by the multimeric adiponectin of 280 kDa or more, and also
quantifying either the reaction intensity of the predetermined anti-adiponectin antibody by the multimeric adiponectin of less than 280 kDa or the reaction intensity of the predetermined anti-adiponectin antibody by the multimeric adiponectin of all the molecular weights contained in the saliva, and
evaluating the salivary adiponectin level, by the ratio of each quantified value.

5. A method for evaluating a salivary adiponectin level, wherein
when a saliva sample is fractionated into human multimeric adiponectin of 280 kDa or more and human multimeric adiponectin of less than 280 kDa, by Western Blotting method under non-denaturing conditions, to extract the band areas exceeding the upper and lower limits of 280 kDa, and react with a specific anti-adiponectin antibody, and a HMW quantitative value obtained by quantifying the reaction intensity in the band area of 280 kDa or more, and MMW/LMW quantitative values obtained by quantifying the reaction intensity in each of the band areas of less than 280 kDa are respectively calculated,
an anti-adiponectin antibody in which the HMW quantitative value is a value that can be significantly evaluated, and the MMW/LMW quantitative values are 1/10 or less of the HMW quantitative value or 0, is selected, and
the salivary adiponectin level is evaluated by the reaction intensity of the selected anti-adiponectin antibody.

6. A kit for measuring a salivary adiponectin level, comprising a reagent of an anti-adiponectin antibody which reacts specifically with multimeric adiponectin of 280 kDa or more in the collected saliva, wherein the salivary adiponectin level is evaluated by the level of the multimeric adiponectin of 280 kDa or more, by the reagent.

7. The method for evaluating a salivary adiponectin level according to claim 2, wherein the saliva sample is a saliva sample collected by a straw.

8. The method for evaluating a salivary adiponectin level according to claim 2, comprising:
reacting saliva with a predetermined anti-adiponectin antibody,
quantifying the reaction intensity of the predetermined anti-adiponectin antibody by the multimeric adiponectin of 280 kDa or more, and also
quantifying either the reaction intensity of the predetermined anti-adiponectin antibody by the multimeric adiponectin of less than 280 kDa or the reaction intensity of the predetermined anti-adiponectin antibody by the multimeric adiponectin of all the molecular weights contained in the saliva, and
evaluating the salivary adiponectin level, by the ratio of each quantified value.

9. The method for evaluating a salivary adiponectin level according to claim 3, comprising:
reacting saliva with a predetermined anti-adiponectin antibody,
quantifying, the reaction intensity of the predetermined anti-adiponectin antibody by the multimeric adiponectin of 280 kDa or more, and also
quantifing either the reaction intensity of the predetermined anti-adiponectin antibody by the multimeric adiponectin of less than 280 kDa or the reaction intensity of the predetermined anti-adiponectin antibody by the multimeric adiponectin of all the molecular weights contained in the saliva, and
evaluating the salivary adiponectin level, by the ratio of each quantified value.

10. The method for evaluating as salivary adiponectin level according to claim 7, comprising:
reacting saliva with a predetermined anti-adiponectin antibody,
quantifying the reaction intensity of the predetermined anti-adiponectin antibody by the multimeric adiponectin of 280 kDa or more, and also
quantifying either the reaction intensity of the predetermined anti-adiponectin antibody by the multimeric adiponectin of less than 280 kDa or the reaction intensity of the predetermined anti-adiponectin antibody by the multimeric adiponectin of all the molecular weights contained in the saliva, and
evaluating the salivary adiponectin level, by the ratio of each quantified value.
